(19) United States
(12) Patent Application Publication
(10) Pub. No.: US 2015/0065945 A1
(43) Pub. Date: Mar. 5, 2015

(54) SPINAL NEUROMODULATION AND ASSOCIATED SYSTEMS AND METHODS

(71) Applicants: Denise ZARINS, Santa Rosa, CA (US); MEDTRONIC ARDIAN LUXEMBOURG SARL, Lexembourg (LU)

(72) Inventors: Denise Zarins, Santa Rosa, CA (US); Sean Ward, Dublin (IE)

(21) Appl. No.: 14/379,841

(22) PCT Filed: Mar. 7, 2013
(86) PCT No.: PCT/US2013/029482

§ 371 Date: Aug. 20, 2014

Related U.S. Application Data
(60) Provisional application No. 61/608,581, filed on Mar. 8, 2012.

(51) Int. Cl. A61B 18/14; A61B 18/18; A61N 7/00; A61K 31/475; A61M 5/14; A61K 31/395; A61K 31/045; A61K 31/05; A61B 18/04; A61B 18/02

(52) U.S. Cl. CPC A61B 18/14; A61B 18/04; A61B 18/18; A61N 7/00; A61B 18/1815; A61B 18/02; A61M 5/14; A61K 31/395; A61K 31/045; A61K 31/05; A61K 31/475; A61B 2018/0044
USPC 604/21; 606/41; 606/27; 606/14; 606/169; 606/33; 606/21; 514/183; 514/724; 514/731; 514/283

(57) ABSTRACT

Methods for treating a patient using therapeutic spinal neuromodulation and associated devices, systems, and methods are disclosed herein. One aspect of the present technology is directed to methods including modulating nerves of one or more targeted organs proximate one or more dorsal root ganglia, stellate ganglia, vertebral ganglia, or cervical ganglia of the nerves using an intravascularly-positioned therapeutic element. One or more measurable physiological parameters corresponding to at least one condition associated with sympathetic activity in the targeted organs and/or central sympathetic activity in the patient can thereby be reduced.

### CROSS-REFERENCE TO RELATED APPLICATION

This application is related to and claims priority to U.S. Provisional Patent Application Ser. No. 61,608,581 filed Mar. 8, 2012, entitled SPINAL NEUROMODULATION AND ASSOCIATED SYSTEMS AND METHODS, the entirety of which is incorporated herein by reference.

### FIELD OF THE INVENTION

The present technology relates generally to spinal neuromodulation and associated systems and methods. In particular, several embodiments of the present technology are directed to modulation of nerves of one or more targeted organs (e.g., the heart or at least one kidney) proximate one or more spinal ganglia associated with the one or more targeted organs to treat at least one condition associated with sympathetic activity (e.g., overactivity or hyperactivity) in the targeted organs and/or central sympathetic activity (e.g., overactivity or hyperactivity).

### BACKGROUND OF THE INVENTION

The sympathetic nervous system (SNS) is a primarily involuntary bodily control system typically associated with stress responses. Fibers of the SNS extend through tissue in almost every organ system of the human body. For example, some fibers extend from the brain, intertwine along the aorta, and branch out to various organs. As groups of fibers approach specific organs, fibers particular to the organs can separate from the groups. Signals sent via these and other fibers can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states.

Overactivity or hyperactivity of the SNS can cause or exacerbate a variety of conditions in organs innervated by sympathetic nerves. There are several currently used methods of treating these conditions by modulating the SNS. These methods include invasive reperfusion therapy followed by a pharmacological regimen of drugs that affect the SNS, use of needle-based devices for methods such as nerve blockade (for example, the practice of injecting of alcohol or local anesthetic with a needle onto or into the stellate ganglia) and image-guided needle radiofrequency (RF) ablation, and open thorascopic surgical or endoscopic methods (such as bilateral cardiac sympathetic denervation (BCSD)). However, all of these methods involve directly accessing the treatment site from outside the body and proximal to the treatment site, and all have significant drawbacks. For example, invasive reperfusion therapy is traumatic to the patient and the use of drugs may have deleterious or unintended side effects that can be difficult to manage. Further, methods such as BCSD are traumatic to the patient and may involve snipping or removing portions of nerves, such as the stellate ganglia, and the toleration of BCSD in humans is still not entirely clear. Additionally, methods such as nerve blockade are difficult to perform, in part because directly accessing the target treatment sites (such as the sympathetic ganglia) with needles and/or needle-like medical devices from outside the body requires absolute precision, and surrounding tissue is easily and commonly damaged. Further, common methods of imaging for needle placement in this type of procedure are imprecise and ineffective. Also, nerve blockade has been shown to be ineffective in the long-term treatment of pain and other conditions, may require multiple treatments, and may result in unintended side effects due to the dissipation of injected drugs to nearby nontarget structures.

It is therefore desired to provide an improved method of modulating spinal nerves of the sympathetic nervous system that is minimally invasive, effective, and reduces the likelihood of patient injury and treatment side effects.

### SUMMARY OF THE INVENTION

The present invention advantageously provides a method and system for neuromodulation of spinal ganglia in the treatment of a variety of conditions involving the sympathetic nervous system. The method may generally include intravascularly positioning a medical device including a therapeutic element in the patient proximate a spinal ganglion, and activating the therapeutic element to modulate the spinal ganglion. For example, the therapeutic element may be positioned proximate a junction of the subclavian artery and the vertebral artery. The method may further include determining a first central sympathetic system activity characteristic value within the patient before modulation of the spinal ganglion, determining a second central sympathetic system activity characteristic value within the patient during or after modulation of the spinal ganglion, comparing the first value to the second value, and calculating a value of change in central sympathetic activity with respect to the central sympathetic system activity characteristic based at least in part on the comparison between the first value and the second value. In some embodiments, the spinal ganglion is the stellate ganglion. The central sympathetic system activity characteristic may be at least one of muscle sympathetic nerve activity and whole-body norepinephrine spillover. In certain embodiments, a predetermined target value of change in central sympathetic activity is determined, the predetermined target change being a reduction of muscle sympathetic nerve activity or whole-body norepinephrine spillover of at least approximately 10% in a period of time after modulating the stellate ganglion. Further, modulating the stellate ganglion may include at least one of at least partially disrupting stellate ganglion nerve function and at least partially regulating stellate ganglion nerve function, and this may be accomplished, for example, by chemically modulating the stellate ganglion or by thermally modulating the stellate ganglion by delivering at least one of radiofrequency energy, optical energy, ultrasound energy (e.g., high intensity focused ultrasound energy or HIFU), microwave energy, pulsed current energy, direct heat energy, or combinations thereof from the therapeutic element to the stellate ganglion, and cryotherapeutically cooling the stellate ganglion with the therapeutic element. For example, delivery of this energy may ablate the stellate ganglion.

Alternatively, the method may include determining a first body system activity characteristic value within the patient before modulation of the spinal ganglion, determining a second body system activity characteristic value within the patient during or after modulation of the spinal ganglion, comparing the first value to the second value, and calculating a value of change in central sympathetic activity with respect to the body system activity characteristic based at least in part on the comparison between the first value and the second value. For example, the body system activity characteristic may be at least one of QT interval, heart rate, cardiac structure, cardiac function, frequency of atrial arrhythmia, frequency of ventricular ectopy, heart rate variability, cardiac norepinephrine spillover, blood pressure, atrial blood flow in the arm, vascular compliance in the arm, perceived pain, occurrence of digital ulceration, severity of digital ulceration, vasospasm, vasoconstriction, occurrence of excess sweating, and severity of excess sweating.

In another embodiment, the method may be for treating a human patient diagnosed with cardiac arrhythmia, and the method may generally include positioning a medical device including a therapeutic element proximate a junction between a subclavian artery and a vertebral artery in a patient, and at least partially inhibiting neural activity in nerves proximate the junction with the therapeutic element. For example, at least partially inhibiting neural activity in the nerves proximate the junction may include at Last partially inhibiting neural activity in a stellate ganglion. The method may further include determining a first cardiac activity characteristic value within the patient before the at least partial inhibition of the neural activity, determining a second cardiac activity characteristic value within the patient during or after the at least partial inhibition of the neural activity, comparing the first value to the second value, and calculating a value of change in central sympathetic system activity with respect to the cardiac activity characteristic based at least in part on the comparison between the first value and the second value., The cardiac activity characteristic may be, for example, severity of cardiac arrhythmia episodes within the patient or frequency of cardiac arrhythmia episodes in the patient. As an example, the value of change in central sympathetic system activity with respect to the cardiac system activity characteristic may be deemed satisfactory when the second value is less than the first value. At least partially inhibiting neural activity may include thermally modulating the nerves, such as by delivering radiofrequency energy to the nerves. Further, this delivery of energy may ablate the nerves.

In another embodiment, the method may be for treating a human patient diagnosed with Raynaud's phenomenon or hyperhidrosis, and the method may generally include positioning a medical device including a therapeutic element proximate a junction between a subclavian artery and a vertebral artery in a patient displaying one or more symptom characteristics, at least partially inhibiting neural activity in a stellate ganglion proximate the junction with the therapeutic element, determining a first symptom characteristic value within the patient before the at least partial inhibition of neural activity in the stellate ganglion, determining a second symptom characteristic value within the patient during or after the at least partial inhibition of neural activity in the stellate ganglion, comparing the first value to the second value, and calculating a value of change in central sympathetic system activity based at least in part on the comparison between the first value and the second value. For example, the one or more symptom characteristics may include digital ulceration, vasoconstriction, vasospasm, pain, and excessive sweating.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 shows a simplified anatomical view of a junction between a subclavian artery and a vertebral artery and nearby structures;

FIG. 2 shows a partially cross-sectional view illustrating neuromodulation at a treatment location proximate the junction of FIG. 2 in accordance with an embodiment of the present technology;

FIG. 3 shows a partially schematic view of a neuromodulation system configured in accordance with an embodiment of the present technology;

FIG. 4A shows a cross-sectional view of a first embodiment of a treatment device positioned proximate the junction of FIG. 2 in accordance with an embodiment of the present technology, the treatment device being in a collapsed configuration;

FIG. 4B shows a cross-sectional view illustrating the treatment device of FIG. 4A, the device being in a deployed configuration;

FIG. 5A shows a partially cross-sectional view illustrating a second embodiment of a treatment device positioned proximate the junction of FIG. 2 in accordance with an embodiment of the present technology, the treatment device being in a collapsed configuration;

FIG. 5B shows a partially cross-sectional view illustrating the treatment device of FIG. 5A, the treatment device being in a deployed configuration;

FIG. 6 shows a partially schematic view of a first embodiment of a treatment device that includes an occlusion element

FIG. 7 shows a partially schematic view of a second embodiment of a treatment device that includes an occlusion element;

FIG. 8A shows a partially schematic view of a third embodiment of a treatment device that includes an occlusion element, the occlusion element being in an unexpanded configuration and the therapeutic element being retracted within the treatment device;

FIG. 8B shows a partially schematic view of the treatment device of FIG. 8A, the occlusion element being in an expanded configuration and the therapeutic element being in contact with tissue; and

FIG. 9 shows a partially schematic view of a third embodiment of a treatment device that includes an occlusion element.

### DETAILED DESCRIPTION OF THE INVENTION

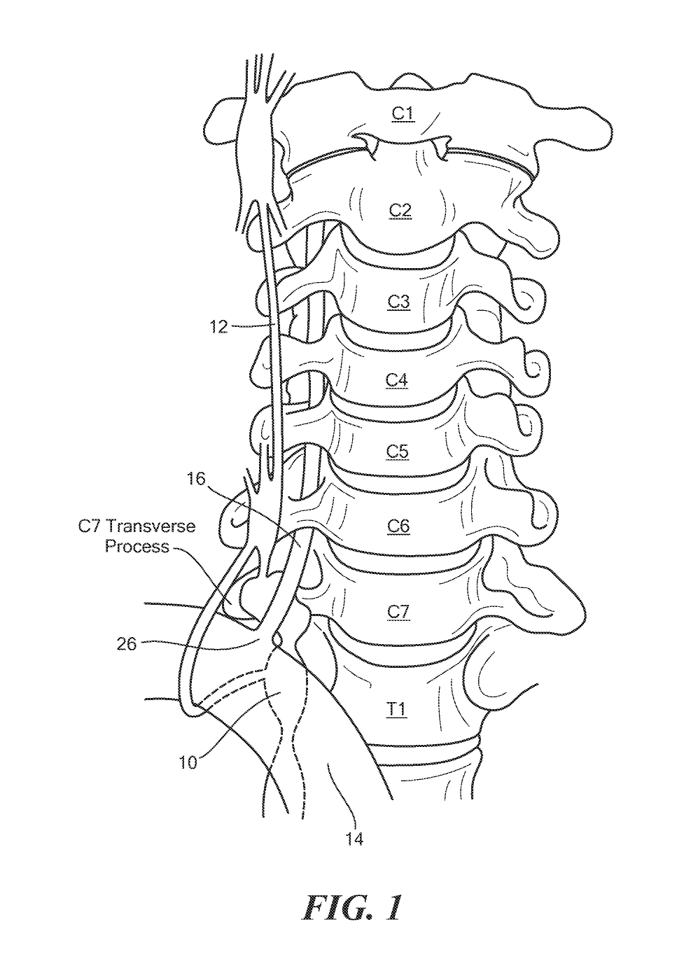

The present technology is generally directed to modulation of nerves of one or more targeted organs (e.g., the heart or at least one kidney) proximate one or more spinal ganglia associated with the one or more targeted organs to treat at least one condition associated with sympathetic activity (e.g., overactivity or hyperactivity) in the targeted organs and/or central sympathetic activity (e.g., overactivity or hyperactivity). Specific details of several embodiments of the present technology are described herein with reference to FIGS. 1-9. Although many of the embodiments arc described herein with respect to cryotherapeutic, electrode-based, transducer-based, and chemical-based approaches, other treatment modalities in addition to those described herein are within the scope of the present technology. Additionally, other embodiments of the present technology can have different configurations, components, or procedures than those described herein. For example, other embodiments can include additional elements and features beyond those described herein or be without several of the elements and features shown and described herein. Generally, unless the context indicates otherwise, the terns “distal” and “proximal” within this disclosure reference a position elative to an operator or an operator's control device. For example, “proximal” can refer to a position closer to an operator or an operator's control device, “distal” can refer to a position that is more distant from an operator or an operator's control device. The headings provided herein are for convenience only.

### Spinal Neuromodulation

For purposes of this disclosure, spinal neuromodulation is the partial or complete incapacitation or other effective disruption or regulation of nerves of one or more targeted organs (e.g., nerves terminating in or originating from the targeted organs or in structures closely associated with the targeted organs) proximate one or more spinal ganglia associated with the one or more targeted organs. For example, at least a portion of the heart is innervated by the stellate ganglia, and at least a portion of the kidneys is innervated by the dorsal root ganglia of the renal nerves. In particular, spinal neuromodulation comprises inhibiting, reducing, blocking, pacing, upregulating, and/or downregulating neural communication along neural fibers, e.g., efferent and/or afferent neural fibers proximate one or more dorsal root ganglia of the neural fibers and/or other spinal ganglia. The methods of spinal neuromodulation described herein may also include neuromodulation of one or more ganglia located proximate the junction between the subclavian artery and the vertebral artery, such as stellate ganglia, as this is expected to have an effect on sympathetic tone. Such incapacitation, disruption, and/or regulation can be long-term (e.g., permanent or for periods of months, years, or decades) or short-term (e.g., for periods of minutes, hours, days, or weeks). Sympathetic neural activity can cause or exacerbate a variety of conditions in organs innervated by sympathetic nerves. Modulation of nerves of these organs is expected to be therapeutically effective for the treatment of such conditions. Modulation of cardiac nerves proximate, for example, the stellate ganglia, is expected to be useful in treating conditions such as cardiac arrhythmias, as these conditions are associated with cardiac sympathetic activity (e.g., overactivity or hyperactivity).

As shown in FIG. 1, a stellate ganglion 10 (or cervicothoracic ganglion or inferior cervical ganglion) is a sympathetic ganglion formed by the fusion of the inferior cervical ganglion and the first thoracic ganglion. The stellate ganglia 10 are located along the sympathetic trunk 12 at the level of C7, anterior to the transverse process of C7, anterior to the neck of the first rib, and just below the subclavian artery 14 proximate the vertebral artery 16. The stellate ganglia are in locations that would be convenient to access from within the arterial vasculature. In most circumstances, the sympathetic ganglia likely supply nerves to structures correlating to C7, C8, and T9 nerves, the inferior cardiac nerve, blood vessels comprising the vertebrobasilar cerebral circulation (vertebral arteries, basilar artery, etc.), subclavian blood vessel tree, and the inferior thyroid artery. The left stellate ganglion may provide more extensive innervation to cardiac structures than the right stellate ganglion. Other conditions that may benefit from spinal neuromodulation include, but are not limited to, Reynaud's disease and hyperhidrosis. The stellate ganglia include efferent sympathetic nerves (which carry nerve impulses away from the central nervous system and toward effectors such as muscles or glands), and may also include some afferent nerves (which carry nerve impulses from receptors toward the central nervous system). In contrast, dorsal root ganglia, located adjacent to the vertebral column, carry sensory nerves into the spinal cord (for example, from the skin, muscle, and other tissues) and include afferent nerves that impact the SNS. So, the neuromodulation site may depend on whether afferent or efferent nerves are a desired target.

Furthermore, sympathetic afferent activity of targeted organs can contribute to central sympathetic tone or drive. Accordingly, spinal neuromodulation may be useful in treating clinical conditions associated with central sympathetic activity (e.g., overactivity or hyperactivity), particularly conditions associated with central sympathetic overstimulation. Sympathetic afferent activity of the kidneys, for example, can have a particularly significant effect on central systemic tone or drive. In some embodiments, modulation of one or more dorsal root ganglia of the renal nerves can be useful in treating clinical conditions associated with central sympathetic activity (e.g., overactivity or hyperactivity). Conditions associated with central sympathetic activity (e.g., overactivity or hyperactivity) include, for example, hypertension and high blood pressure, among other conditions. Some or all of the functionality of the sympathetic nerves of one or more targeted organs can be redundant or otherwise unnecessary for general health. Accordingly, in some patients, reducing sympathetic drive in one or more targeted organs, reducing central sympathetic drive, and/or other benefits from spinal neuromodulation can outweigh the complete or partial loss of sympathetic-nerve functionality in the targeted organs.

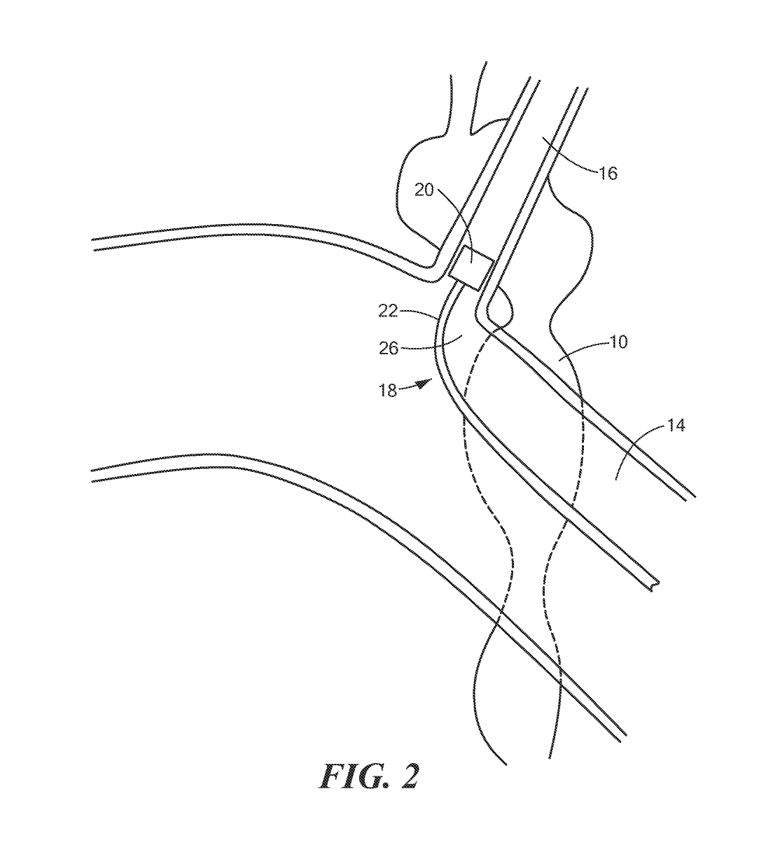

Spinal neuromodulation in accordance with embodiments of the present technology can be electrically-induced, thermally-induced, chemically-induced, or induced in another suitable manner or combination of manners at one or more suitable treatment locations during a treatment procedure. For example, the purposeful application of radiofrequency (RF) energy, microwave energy, laser energy, ultrasound energy (e.g., high intensity focused ultrasound or HIFU energy), cryotherapeutic energy, direct heat energy, chemicals (e.g., drugs or other agents), pulsed current, or combinations thereof to tissue at a treatment location can induce one or more desired effects at the treatment location, e.g., broadly across the treatment location or at localized regions of the treatment location. In FIG. 2, for simplicity, a treatment device 18 is shown with an unspecified therapeutic element 20 located at the distal portion 22 of the treatment device 18 that may be configured for any treatment modality, such as those listed above. For example, the therapeutic element 20 may be an expandable element such as a RF balloon, cryotherapy balloon, a basket or helical structure bearing one or more electrodes, expandable electrode carrier arms, and the like. Activating the therapeutic element 20 may include, for example, heating, cooling, stimulating, or applying another suitable treatment modality at the treatment location. Alternatively, the therapeutic element 20 may be, for example, one or more electrodes or one or more microneedles (such as for the intravascular application of chemical-based treatment) coupled to the distal portion 22 of a fixed-diameter treatment device 18, such as a focal catheter. Additionally, the treatment device 18 may include an occlusion element 24 to prevent emboli generated from the treatment site from traveling to other parts of the patient's body, such as the brain.

In some embodiments, a treatment procedure can include applying a suitable treatment modality at a treatment location in a testing step followed by a treatment step. The testing step, for example, can include applying the treatment modality at a lower intensity and/or for a shorter duration than during the treatment step. This can allow an operator to determine (e.g., by neural activity sensors and/or patient feedback) whether nerves proximate the treatment location are suitable for modulation. Performing a testing step can be particularly useful for treatment procedures in which targeted nerves are closely associated with nerves that could cause undesirable side effects if modulated during a subsequent treatment step.

The treatment location can be proximate (e.g., at or near) a vessel or chamber wall (e.g., a wall of a subclavian artery 14, a vertebral artery 16, a junction 26 between a subclavian artery 14 and a vertebral artery 16, a vascular structure proximate one or more dorsal root ganglia of renal nerves, and/or another suitable structure), and the treated tissue can include tissue proximate the treatment location. For example, with regard to a junction 26 between a subclavian artery 14 and a vertebral artery 16 (as shown in FIG. 2), a treatment procedure can include modulating nerves in a stellate 10 and/or thoracic ganglion, which are proximate the junction 26. As a non-limiting example, depending on the location of the target nerves (for example, a stellate ganglion), the treatment device 18 may be positioned in the subclavian artery 14 or vertebral artery 16 proximate the junction 26 thereof. Further, a treatment procedure may include the treatment of one or more ganglia along the sympathetic trunk 12, for example, the stellate ganglia 10. Additionally, the treatment may be tailored based on serially treating one target location at a time until a desired clinical effect is achieved. As a non-limiting example, a desired clinical effect of neuromodulation of renal dorsal root ganglia may be a reduction in systolic blood pressure of, for example, approximately 10 mmHg to approximately 20 mmHg, such as 15 mmHg. Furthermore, a treatment location can be proximate nerves that are predominately afferent (such as the dorsal root ganglia of, for example, the renal nerves) or predominantly efferent (such as the stellate ganglia). Such treatment locations can be more available within or proximate the spinal anatomy than at other anatomical positions more distant from the spine. Selecting treatment location according to the predominant anatomical positions of afferent or efferent nerves can allow the afferent or efferent nerves to be preferentially modulated. This can be particularly useful when afferent activity alone or efferent activity alone is primarily or solely responsible for a condition to be treated.

Effects of intravascular Spinal Neuromodulation

Current methods of modulating sympathetic nerves involve assessing the treatment site form outside the body and targeting the nerves directly (for example, directly injecting anesthetic into a stellate ganglion 10, such as in a nerve block). As described above, these methods are difficult and can easily cause patient injury and unintended side effects, and may require multiple treatments or provide only temporary results. Intravascular spinal neuromodulation methods of the present invention overcome these disadvantages by providing a method that is minimally invasive, generates fewer unintended side effects, requires fewer treatments, and/or has a more significant clinical effect in the treatment of conditions such as Raynaud's phenomenon, hyperhidrosis, certain cardiac arrhythmias (e.g., ventricular tachycardia), hypertension, and high blood pressure, among others.

### 1. Neuromodulation of Stellate Ganglia

A. Cardiac Conditions

Current research, in pigs and dogs, shows that stellate ganglion blockade reduces catecholamine release at the level of the myocardium. Catecholamines (for example, epinephrine and norepinephrine) are known to increase the propensity for both atrial and ventricular arrhythmia. Stellate ganglion blockade is used to treat rare or intractable forms of atrial tachycardia, and the method reduces catecholamine release at the level of the myocardium (according to studies conducted on pigs and dogs). However, current methods of performing stellate ganglion blockade have significant drawbacks, such as patient injury and trauma and unintended side effects. Intravascular modulation of sympathetic innervation to the heart via the stellate ganglia as disclosed herein may be a better alternative for blocking or otherwise modulating the effect of the above-described neurohormones, thereby providing beneficial effects in cases of atrial arrhythmia, ventricular arrhythmia, and/or heart failure.

Sympathetic activity is found to be elevated in cases of heart failure, and conditions such as ventricular tachycardia appear to be directly related to the degree of cardiac sympathetic innervation. Additionally, sympathetic input to the heart may influence ventricular remodeling following myocardial infarction, which may lead to heart failure. Still further, research shows that stimulation of the stellate ganglia increases QT interval (that is, the time between the start of the Q wave and the end of the T wave in the cardiac electrical cycle). In general, the QT interval represents the electrical depolarization and repolarization of the left and right ventricles, and a longer QT interval is associated with an increased propensity to ventricular arrhythmia. Current methods of treatment of long QT syndrome include removal of the first four or five thoracic sympathetic ganglia (left stellate ganglionectomy) or treatment with drugs such as beta-blockers or other anti-arrhythmics, and implantable cardioverter-defibrillators. However, for the reasons described hereinabove, these methods have drawbacks that are overcome by the intravascular stellate ganglia neuromodulation methods of the present invention, which are expected to have a beneficial effect in cases of cardiac arrhythmias because stellate ganglia provide sympathetic input to regions of the myocardium.

B. Conditions Affecting the Extremities

There are many conditions that can adversely affect the extremities of the body, in particular the vasculature. For example, Raynaud's phenomenon involves abnormal sensitivity of small arteries and arterioles to vasoconstriction stimuli, and can involve vasopasm and vasoconstriction of the digital arteries causing pallor with cyanosis and/or rubor. Raynaud's can be primary (idiopathic, also called Raynaud's disease), in which it is not associated with other diseases, or secondary to several diseases or conditions (called Raynaud's syndrome). Complications associated with this condition include digital ulcers, possibly leading to amputation. Raynaud's phenomenon currently may be treated with drugs (e.g., angiotensin II inhibitors, selective serotonin reuptake inhibitors, phosphodiesterase-5 inhibitors, nitrates, and/or prostacyclin agonists), the use of many of which is limited by adverse side effects. Research indicates that in severe cases, such as in patients with systemic sclerosis, stellate ganglion blockade or sympathectomy results in patient improvement. Intravascular stellate ganglia neuromodulation methods of the present invention, which are less invasive and more effective than pharmaceutical stellate ganglion blockade, are expected to be effective in the treatment of Raynaud's phenomenon. For example, intravascular stellate ganglia neuromodulation can be expected to reduce the frequency and/or severity of ulceration and pain associated with this condition.

Further, research indicates that stellate ganglion blockade is effective in the treatment of upper extremity ischemia by reducing sympathetic drive to the upper extremities and thus improving regional blood flow to the upper limbs. Still further, research shows that hyperhidrosis or excessive sweating of, for example, the hands (which is under the control of the SNS), is expected to be manageable by manipulating sympathetic drive, such as by stellate ganglion blockade. However, current stellate ganglion blockade methods involve directly accessing the nerves from outside the body, and may be traumatic to the patient and/or cause adverse side effects. Intravascular stellate ganglia neuromodulation methods of the present invention, which are less invasive and more effective than stellate ganglion blockade, are expected to be effective in the treatment of certain conditions affecting the extremities. For example, intravascular stellate ganglia neuromodulation can be expected to reduce or prevent excessive sweating.

C. Chronic Pain Disorders

Complex regional pain syndrome (CRPS) is an extremely painful condition of the limbs, and usually arises after trauma. Research shows that stellate ganglion blockade and RF neurolysis or destruction of nerve tissue are effective in reducing pain associated with this condition. In fact, RF neurolysis of the stellate ganglia has been shown to provide pain relief over a significantly longer period of time than stellate ganglion blockade. Additionally, there is data that suggests that stellate ganglion blockade can limit the severity and course of pain experienced in facial post-herpetic neuralgia, which is a skin disease caused by reactivation of a latent varicella zoster virus in dorsal root ganglia. However, current treatment methods involve directly accessing the nerves from outside the body, and may be traumatic to the patient and/or cause adverse side effects. Intravascular stellate ganglia neuromodulation methods of the present invention (especially RF neuromodulation), which are less invasive and more effective than stellate ganglion blockade, are expected to have beneficial effects in the treatment of these disorders.

D. Hot Flashes

Hot flashes (or hot flushes) occur in about 80% to about 90% of postmenopausal women. In about 20% of postmenopausal women, hot flashes may last for up to 15 years or longer. The exact pathophysiology of flushing is not yet filly understood, but it includes an acute vasomotor activity that is influenced by estrogen. It has been hypothesized that one of the problems that cause hot flushes is dysregulation of the body's response to temperature cues. It is also believed that the stellate ganglia may affect parts of the central nervous system responsible for thermoregulation and that stellate ganglion blockade may reset inappropriate thermoregulatory responses. However, as discussed above, stellate ganglion block involves directly accessing the nerves from outside the body to inject materials such as alcohol or anesthetics, and may be traumatic to the patient and/or cause adverse side effects. Intravascular stellate ganglia neuromodulation methods of the present invention, which are less invasive and more effective than stellate ganglion blockade, are expected to be effective in the treatment of hot flashes.

### 2. Neuromodulation of Dorsal Root Ganglia of Renal Nerves

Research indicates that modulation of renal nerves is a safe and effective treatment for conditions associated with overactivity or hyperactivity of the SNS, such as hypertension. Current methods involve the modulation of nerves within the adventitia of the renal artery. Whereas neuromodulation of the renal nerves within the renal arteries is effective in treating conditions such as hypertension and high blood pressure, intravascular neuromodulation of the dorsal root ganglia of the renal nerves may have a significant clinical effect in the treatment of alternate conditions, such as cardiac arrhythmias, Raynaud's phenomenon, and hyperhidrosis.

### Selected Examples of Neuromodulation Systems

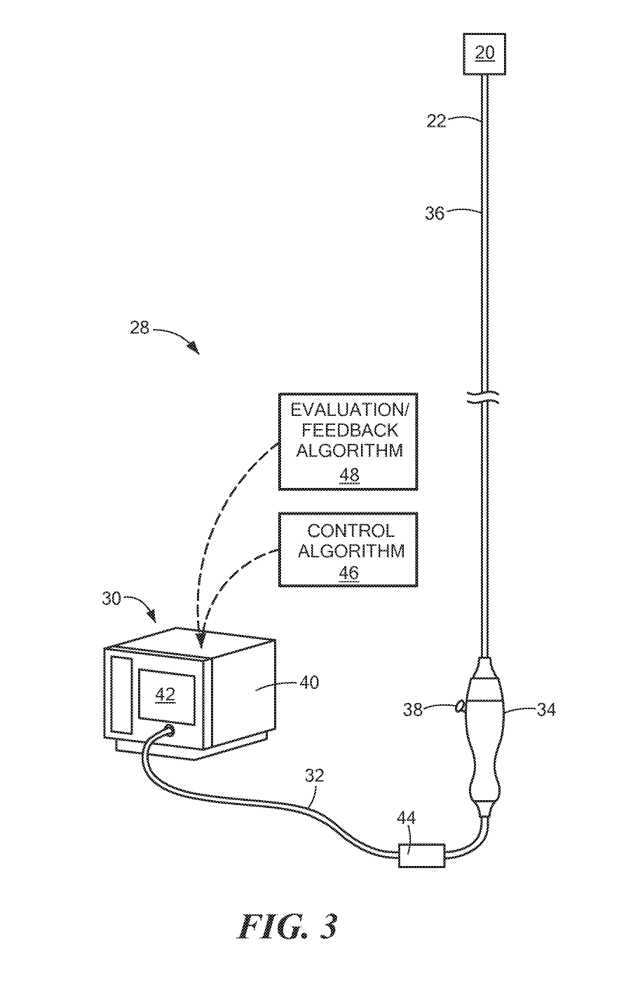

FIG. 3 is a partially schematic diagram illustrating a neuromodulation system 28 (“system 28”) configured in accordance with an embodiment of the present technology. The system 28 can include a treatment device 18, an energy source or console 30 (e.g., an RF energy generator, a cryotherapy console, etc.), and a cable 32 extending between the treatment device 18 and the energy source or console 30. The treatment device 18 can include a handle 34, a therapeutic element 20, and an elongated shaft 36 extending between the handle 34 and the therapeutic element 20. The shaft 36 can be configured to locate the therapeutic element 20 intravascularly at a treatment location (e.g., in or near a subclavian artery 14, a vertebral artery 16, a junction 26 between a subclavian artery 14 and a vertebral artery 16, a vascular structure proximate one or more dorsal root ganglia of renal nerves, and/or another suitable structure), and the therapeutic element 20 can be configured to provide or support therapeutically-effective neuromodulation at the treatment location. In some embodiments, the shaft 36 and the therapeutic element 20 can be 3, 4, 5, 6, or 7 French or another suitable size. Furthermore, the shaft 36 and the therapeutic element 20 can be partially or fully radiopaque and/or can include radiopaque markers corresponding to measurements, e.g., every 5 cm.

Intravascular delivery can include percutaneously inserting a guide wire within the vasculature and moving the shaft 36 and the therapeutic element 20 along the guide wire until the therapeutic element 20 reaches the treatment location. For example, the shaft 36 and the therapeutic element 20 can include a guide-wire lumen (not shown) configured to receive the guide wire in an over-the-wire (OTW) or rapid-exchange (RX) configuration. Other body lumens (e.g., ducts or internal chambers) can be treated, for example, by non-percutaneously passing the shaft 36 and therapeutic element 20 through externally accessible passages of the body or other suitable methods. In some embodiments, a distal end of the therapeutic element 20 can terminate in an atraumatic rounded tip or cap (not shown). The treatment device 18 can also be a steerable or non-steerable catheter device configured for use without a guide wire.

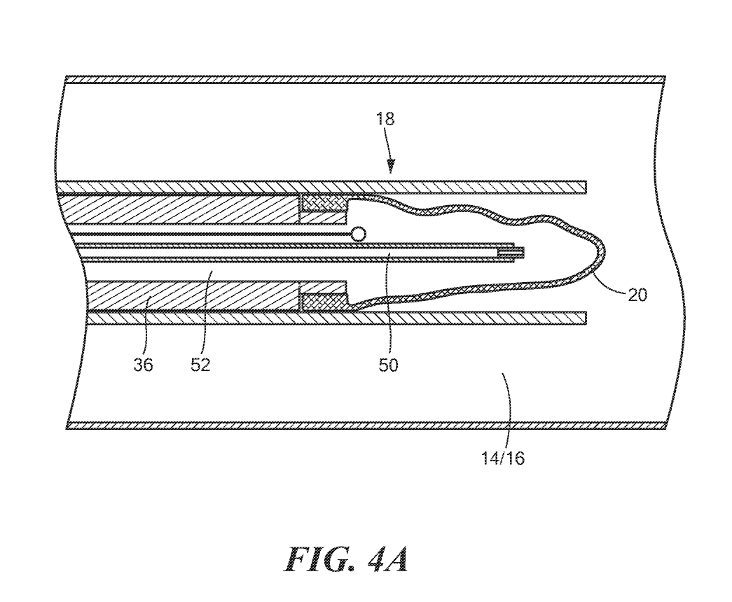

The therapeutic element 20 can have a single state or configuration, or it can be convertible between a plurality of states or configurations (for example, as shown and described in FIGS. 4A-9). For example, the therapeutic element 20 can be configured to deflect into contact with a vessel wall in a delivery state. The therapeutic element 20 can be converted (e.g., placed or transformed) between the delivery and deployed states via remote actuation, e.g., using an actuator 38 of the handle 34. The actuator 38 can include a knob, a pin, a lever, a button, a dial, or another suitable control component. In other embodiments, the therapeutic element 20 can be transformed between a delivery and deployed states using other suitable mechanisms or techniques.

In some embodiments, the energy source or console 30 may be configured to control, monitor, supply, or otherwise support operation of the treatment device 18. In other embodiments, the treatment device 18 may be self-contained and/or otherwise configured for operation without connection to the energy source or console 30. As shown in FIG. 1, the energy source or console 30 may include a primary housing 40 having a display 42. The system 28 may include a control device 44 along the cable 32 configured to initiate, terminate, and/or adjust operation of the treatment device 18 directly and/or via the energy source or console 30. In other embodiments, the system 28 may include another suitable control mechanism. For example, the control device 44 may be incorporated into the handle 34. The energy source or console 30 may be configured to execute an automated control algorithm 46 and/or to receive control instructions from an operator. Furthermore, the energy source or console 30 may be configured to provide feedback to an operator before, during, and/or after a treatment procedure via the display 42 and/or an evaluation/feedback algorithm 48. In some embodiments, the energy source or console 30 may include a processing device (not shown) having processing circuitry, e.g., a microprocessor. The processing device may be configured to execute stored instructions relating to the control algorithm 46 and/or the evaluation/feedback algorithm 48. Furthermore, the energy source or console 30 may be configured to communicate with the treatment device 18, e.g., via the cable 32. For example, the therapeutic element 20 of the treatment device 18 can include a sensor (not shown) (e.g., a recording electrode, a temperature sensor, a pressure sensor, or a flow rate sensor) and a sensor lead (not shown) (e.g., an electrical lead or a pressure lead) configured to carry a signal from the sensor to the handle 34. The cable 32 may be configured to carry the signal from the handle 34 to the energy source or console 30.

The energy source or console 30 may have different configurations depending on the treatment modality of the treatment device 18. For example, when the treatment device 18 is configured for electrode-based or transducer-based treatment, the energy source or console 30 may include an energy generator (not shown) configured to generate RF energy, pulsed RF energy, microwave energy, optical energy, focused ultrasound energy (e.g., high-intensity focused ultrasound energy), direct heat energy, or another suitable type of energy. in some embodiments, the energy source or console 30 may include an RF generator operably coupled to one or more electrodes (not shown) of the therapeutic element 20. When the treatment device 18 is configured for cryotherapeutic treatment, the energy source or console 30 may include a refrigerant reservoir (not shown) and may be configured to supply the treatment device 18 with refrigerant, e.g., pressurized refrigerant in liquid or substantially liquid phase. Similarly, when the treatment device 18 is configured for chemical-based treatment, the energy source or console 30 may include a chemical reservoir (not shown) and may be configured to supply the treatment device 18 with the chemical. In some embodiments, the treatment device 18 may include an adapter (not shown) (e.g., a luer lock) configured to be operably coupled to a syringe (not shown). The adapter may be fluidly connected to a lumen (not shown) of the treatment device 18, and the syringe may be used, for example, to manually deliver one or more chemicals to the treatment location, to withdraw material from the treatment location, to inflate a balloon (for example, as shown in FIGS. 4A and 4B) of the therapeutic element 20, to deflate a balloon of the therapeutic element 20, or for another suitable purpose. In other embodiments, the energy source or console 30 may have other suitable configurations.

Referring now to FIGS. 4-9, specific and non-limiting examples of treatment devices 18 are shown. In these figures, the treatment device 18 is shown at or near the junction 26 between the subclavian artery 14 and vertebral artery 16, which is a convenient location for performing neuromodulation of the stellate ganglia. FIGS. 4A and 4B show a treatment device 18 that includes an expandable therapeutic element 20. Although not specifically shown, the treatment device 18 can be positioned proximate the junction 26, for example, within the subclavian artery 14 or the vertebral artery 16. For simplicity, the hollow anatomical feature shown in FIGS. 4A and 4B is referred to as 14/16. The expandable therapeutic element 20 shown in FIGS. 4A and 4B may include one or more electrodes for delivering one or more types of energy, such as RF energy. As a non-limiting example, the expandable element 20 may be composed of a conductive mesh or electrode array, or a cryoballoon may include an electrode array on its outer surface. Electrode-based treatment can include delivering electrical energy and/or another form of energy to tissue at a treatment location to stimulate and/or heat the tissue in a manner that modulates neural function. For example, sufficiently stimulating and/or heating at least a portion of a sympathetic nerve can slow or potentially block conduction of neural signals to produce a prolonged or permanent reduction in sympathetic activity. A variety of suitable types of energy can be used to stimulate and/or heat tissue at a treatment location. For example, neuromodulation in accordance with embodiments of the present technology can include delivering RF energy, pulsed RE energy, microwave energy, optical energy, focused ultrasound energy (e.g., high-intensity focused ultrasound energy), direct heat, or another suitable type of energy alone or in combination. In some embodiments, energy can be used to reduce damage to non-targeted tissue when targeted tissue adjacent to the non-targeted tissue is cooled.

Heating effects of electrode-based or transducer-based treatment can include ablation and/or non-ablative alteration or damage, e.g., via sustained heating and/or resistive heating (for example, using energy modalities such as RF energy). For example, a treatment procedure can include raising the temperature of target neural fibers to a target temperature above a first threshold to achieve non-ablative alteration, or above a second, higher threshold to achieve ablation. In some embodiments, the target temperature can be higher than about body temperature (e.g., about 37° C.) but less than about 45° C. for on-ablative alteration, and the target temperature can be higher than about 45° C. for ablation. Heating tissue to a temperature between about body temperature and about 45° C. can induce non-ablative alteration, for example, via moderate heating of target neural fibers or of vascular structures that perfuse the tine target neural fibers. In cases there vascular structures are affected, the target neural fibers can be denied perfusion resulting in necrosis of the neural tissue. Heating tissue to a target temperature higher than about 45° C. (e.g., higher than about 60° C.) can induce ablation, for example, via substantial heating of target neural fibers or of vascular structures that perfuse the target fibers. In some patients, it can be desirable to heat tissue to temperatures that are sufficient to ablate the target neural fibers or the vascular structures, but that are less than about 90° C., e.g., less than about 85° C., less than about 80° C., or less than about 75° C. Other embodiments can include heating tissue to a variety of other suitable temperatures.

Further, the use of RF energy can be advantageous in that, unlike cryotherapy, no additional fluid sources may be needed, thus allowing for a smaller and simpler console. Likewise, treatment device may be used that does not require an inflation medium and eliminates the risk of leakage and patient exposure to refrigerants or other materials. RF energy may also be delivered such that an area below the surface of the tissue (e.g., an artery lumen) is targeted. This may be particularly important for the intravascular neuromodulation methods described herein, because extravascular targets such as the stellate ganglia and dorsal root ganglia of renal nerves may be treated while minimizing trauma to the wall of the vasculature (e.g., subclavian artery 14 or vertebral artery 16) in which the treatment device is positioned.

Additionally or alternatively, the expandable therapeutic element 20 may be cryotherapy balloon defining a lumen in which cryogenic fluid may circulate. The treatment device 18 may also include a fluid injection lumen 50 and a fluid return lumen 52. Although not shown, a treatment device 18 configured for cryotherapy may include a needle-like probe used under fluoroscopic, computed tomography (CT), or another imaging technique. Other known cryotherapeutic devices may also be used.

Cryotherapeutic treatment can include cooling tissue at a treatment location in a manner that modulates neural function. For example, sufficiently cooling at least a portion of a sympathetic nerve can slow or potentially block conduction of neural signals to produce a prolonged or permanent reduction in sympathetic activity. This effect can occur as a result of cryotherapeutic tissue damage, which can include, for example, direct cell injury (e.g., necrosis), vascular injury (e.g., starving cells from nutrients by damaging supplying blood vessels), and/or sublethal hypothermia with subsequent apoptosis. Exposure to cryotherapeutic cooling can cause acute cell death (e.g., immediately after exposure) and/or delayed cell death, e.g., during tissue thawing and subsequent hyperperfusion. Neuromodulation using a cryotherapeutic treatment in accordance with embodiments of the present technology can include cooling a structure proximate an inner surface of a vessel or chamber wall such that tissue is effectively cooled to a depth where sympathetic nerves reside. For example, a cooling assembly of a cryotherapeutic device can be cooled to the extent that it causes therapeutically-effective, cryogenic neuromodulation. In some embodiments, a cryotherapeutic treatment modality can include cooling that is not configured to cause neuromodulation. For example, the cooling can be at or above cryogenic temperatures and can be used to control neuromodulation via another treatment modality, e.g., to reduce damage to non-targeted tissue when targeted tissue adjacent to the non-targeted tissue is heated.

Neuromodulation using cryotherapeutic treatment can be beneficial relative to neuromodulation using other treatment modalities. For example, rapidly cooling tissue can provide an analgesic effect such that cryotherapeutic treatment can be less painful than other treatment modalities. Neuromodulation using cryotherapeutic treatment can therefore require less analgesic medication to maintain patient comfort during a treatment procedure compared to neuromodulation using other treatment modalities. Additionally, reducing pain can reduce patient movement and thereby increase operator success and/or reduce procedural complications. Cryogenic cooling also typically does not cause significant collagen tightening, and therefore is not typically associated with vessel stenosis. In some embodiments, cryotherapeutic treatment can include cooling at temperatures that can cause therapeutic elements 20 to adhere to moist tissue. This can be beneficial because it can promote stable, consistent, and continued contact during treatment. The typical conditions of treatment can make this an attractive feature because, for example, patients can move during treatment, catheters associated with therapeutic elements 20 can move, and/or respiration can cause the spine to rise and fall and thereby move the vasculature around the spine. In addition, blood flow is pulsatile and can cause structures associated with the spine to pulse. Cryogenic adhesion also can facilitate intravascular positioning, particularly in relatively small structures (e.g., relatively short arteries) in which stable intravascular positioning can be difficult to achieve. Thus, any of a variety of energy modalities (including RF therapy or cryotherapy) may be suitable for intravascular neuromodulation. A choice as to the energy modality may be made based on considerations such as patient anatomy, condition being treated, target treatment site, system requirements, preferred treatment device, and the like.

As is further shown in FIGS. 4A and 4B, the treatment device 18 may be delivered to a target treatment site in a collapsed or low-profile configuration (delivery configuration, as shown in FIG. 4A), wherein the expandable therapeutic element 20 is disposed within the shaft 36 of the treatment device 18. Once at the target treatment site, the treatment device may be transitioned for neuromodulation of the target tissue such that the expandable therapeutic element 20 is expanded or deployed and in contact with or proximate at least a portion of the target site (as shown in FIG. 4B). The therapeutic element 20 may be in contact with or proximate at least a portion of the inner wall of the vasculature when in the deployed configuration. Further, the therapeutic element 20 may be in contact with the entire or substantially the entire inner circumference of the portion of vasculature when in the deployed configuration, as shown in FIG. 4B, which may create fully-circumferential lesions or partially circumferential lesions.

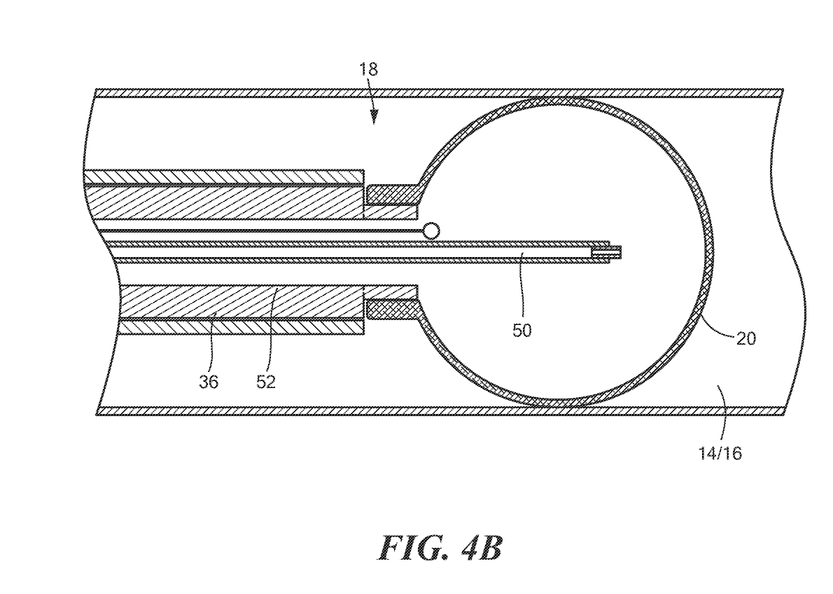

FIGS. 5A and 5B show an alternative embodiment of a treatment device 18 having an expandable therapeutic element 20. The treatment device 18 is shown proximate the junction n 26 between the subclavian artery 14 and vertebral artery 16, with the treatment element 20 being within the vertebral artery 16. However, the device 18 may be positioned at any suitable location. Like the treatment device 18 of FIGS. 4A and 4B, the treatment device 18 of FIGS. 5A and 5B may be delivered to a target treatment site in a collapsed or low-profile configuration in which the expandable therapeutic element 20 is disposed within the shaft 36 of the device (as shown in FIG. 5A). Likewise, the treatment device 18 may be transitioned for neuromodulation of the target tissue such that the expandable element 20 is expanded or deployed and in contact with or proximate at least a portion of the target site (as shown in FIG. 5B). The therapeutic element 20 of the device 18 may have a helical shape that expands when the therapeutic element 20 is advanced out of the device shaft 36 and/or the device shaft 36 or sheath is retracted from the therapeutic element 20, which may create helically patterned lesions (for example, lesions with either discrete lesions in a helical pattern or continuous helical lesions). Further, the therapeutic element 20 may include one or more electrodes 54 configured for use with any of a variety of treatment modalities, including the application of RF energy.

Further, although not shown, a treatment device 18 may be configured for neuromodulation using a chemical-based treatment modality alone or in combination with another treatment modality. Neuromodulation using chemical-based treatment can include delivering one or more chemicals (e.g., drugs or other agents) to tissue at a treatment location in a manner that modulates neural function. The chemical, for example, can be selected to affect the treatment location generally or to selectively affect some structures at the treatment location over other structures. For example, the chemical can be guanethidine, ethanol, phenol, a neurotoxin, vincristine, or another suitable agent selected to alter, damage, or disrupt nerves. A variety of suitable techniques can be used to deliver chemicals to tissue at a treatment location. In an intravascular example, a catheter can be used to intravascularly position a therapeutic element 20 including a plurality of small, flexible needles that can be retracted or otherwise unexposed prior to deployment. In other embodiments, a chemical can be introduced into tissue at a treatment location via simple diffusion through a vessel wall, electrophoresis, or another suitable mechanism. Similar techniques can be used to introduce chemicals that are not configured to cause neuromodulation, but rather to facilitate neuromodulation via another treatment modality. Examples of such chemicals include, but are not limited to, anesthetic agents and contrast agents.

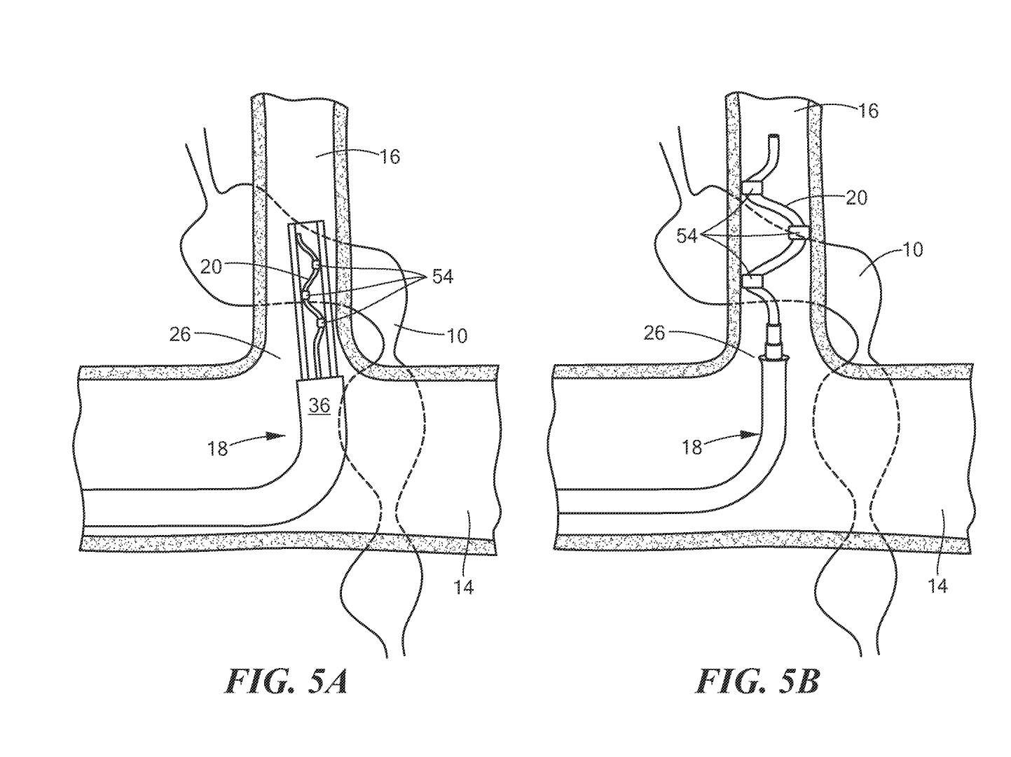

Referring now to FIGS. 6-9, various non-limiting embodiments of a treatment device 18 including an occlusion element 24 are shown. When neuromodulating tissue within the vasculature, especially if the neuromodulation includes ablation, there is a risk that charred blood, cellular components, tissue, or other thrombogenic debris or emboli will travel to the brain, causing a stroke. This risk is especially present when a treatment is performed proximate major arteries leading to the brain, such as the vertebral artery 16. It therefore may be desired to perform the intravascular spinal neuromodulation procedures described herein using a treatment device 18 that includes one or more filters or occlusion elements 24 to prevent debris from entering, for example, the vertebral artery 16. The device embodiments in FIGS. 6-9 are shown in a partially schematic view, because the type of each component may vary. For example, the occlusion element 24 may be a balloon, a mesh, a basket, etc.

FIG. 6 shows a first embodiment of a treatment device 18 that includes an occlusion element 24. The treatment device 18 may generally include a distal first expandable therapeutic element 20 for treatment, such as a conductive expandable mesh or array, and a proximal second expandable element 24 (occlusion element), such as an occlusion balloon or non-conductive expandable mesh, for occlusion of one or more arteries. Alternatively, the expandable therapeutic element 20 may be a cryoballoon usable for cryotherapeutic treatment. If an occlusion balloon is used as the occlusion element 24, the balloon may be substantially toroidal or include one or more channels therethrough to allow the flow of blood through the balloon while still blocking, for example, the vertebral artery 16. For example, the occlusion element 24 may be a balloon that is wrapped about an outer surface of the device shaft 36 in a delivery configuration. When in an expanded configuration, the balloon may be inflated to a toroidal or cylindrical shape that s suitable for blocking, for example, the opening of the vertebral artery 16 from within the subclavian artery 14, while still allowing blood flow through the subclavian artery 14. At least a portion of the balloon may be affixed to the device shaft 36 and in communication with one or more inflation lumens. For example, a portion of the interior of the balloon may be directly affixed to shaft 36, or the balloon may include a tongue or strip of balloon material that is affixed to the shaft 36. Alternatively, the balloon may be affixed to an outer sheath that is slidably disposable about the device shaft 36, allowing the therapeutic element 20 to be positioned relative to the location of the balloon. Other types of occlusion elements 24, such as an expandable mesh, may similarly be coupled to the treatment device 18, albeit without being in communication with one or more inflation lumens.

As shown in FIG. 6 an occlusion element 24 (such as an occlusion balloon) may be positioned such that the vertebral artery 16 is substantially occluded when a therapeutic element 20 (such as an expandable conductive mesh) is used to modulate nerves proximate the subclavian artery 14. In the non-limiting configuration shown in FIG. 6, the device 18 may be positioned within the subclavian artery 14 via radial or brachial access. Blood flow within the subclavian artery 14 is in the direction indicated by arrow 56, or in the direction from the therapeutic element 20 toward the vertebral artery 16 and occlusion element 24. Thus, neuromodulation occurs upstream of the occlusion element 24 and vertebral artery 16.

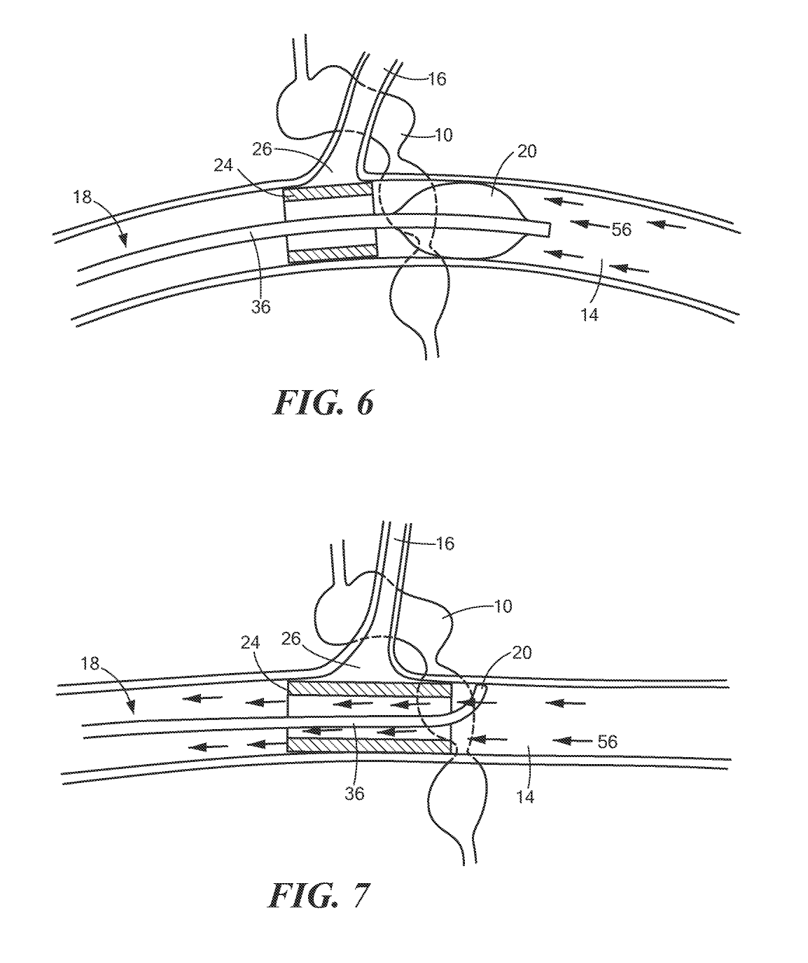

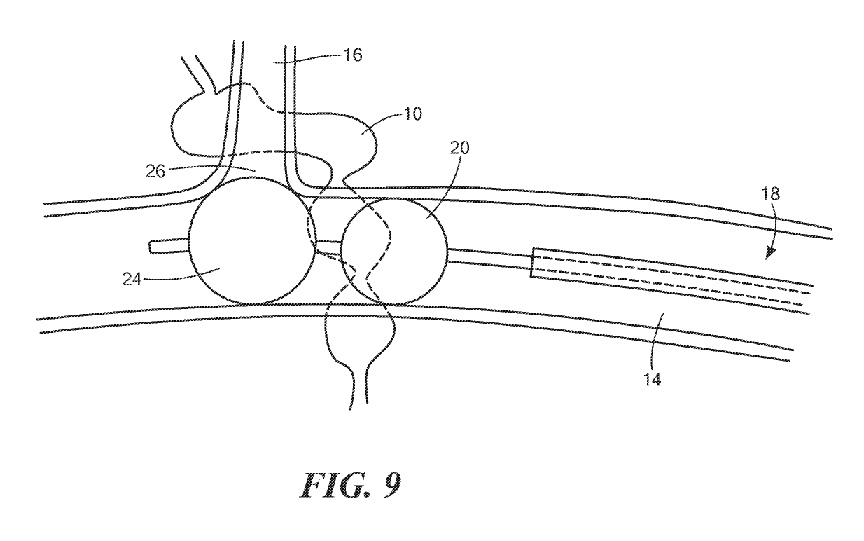

Referring now to FIG. 7, a second embodiment of a treatment device 18 that includes an occlusion element 24 is shown. The treatment device 18 of FIG. 9 may include a proximal occlusion element 24 like that shown and described in FIG. 6. However, the device 18 may have a fixed diameter (except for the occlusion element 24), and the therapeutic element 20 may be one or more electrodes 54 coupled to a distal portion of the device. The one or more electrodes may be configured to deliver any of a variety of treatment modalities, including the application of RF energy. The electrodes 54 may be in communication with one or more energy generators (for example, an RF generator or microwave generator), thermoelectric coolers, or one or more cryogenic fluid sources, and/or the console 30. Additionally or alternatively, the therapeutic element 20 may include one or more LED or laser diodes to neuromodulate the tissue using optical energy (not shown). In the non-limiting configuration shown in FIG. 7, the device 18 may be positioned within the subclavian artery 14 via radial or brachial access. Blood flow within the subclavian artery is in the direction indicated by arrow 56, or in the direction from the therapeutic element 20 toward the vertebral artery 16 and occlusion element 24.

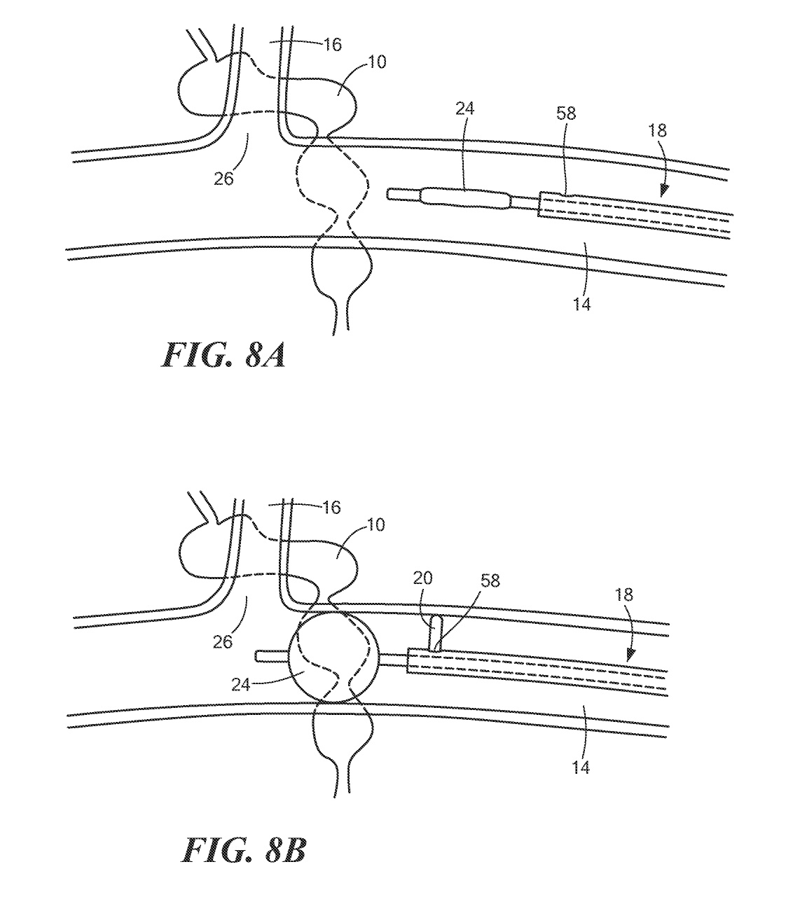

Referring now to FIGS. 8A-9, third and fourth embodiments of a treatment device 18 that includes an occlusion element 24 are shown. As shown and described in FIGS. 6 and 7, the treatment device 18 may be used to modulate nerves (for example, stellate ganglia) proximate the junction 26 of the subclavian artery 14 and vertebral artery 16 via brachial or radial access. In contrast, however, the treatment device 18 of the configurations shown in FIGS. 8A-9 may be positioned proximate the junction 26 of the subclavian artery 14 and vertebral artery 16 via femoral access. Like the treatment devices 18 of FIGS. 6 and 7, the treatment device 18 of FIGS. 8A-9 may include an occlusion element 24 that spans the entire circumference of the subclavian artery 14, so as to prevent debris from the neuromodulation site from entering the vertebral artery 16.

In the third embodiment shown in FIGS. 8A and 8B, the shaft 36 of the treatment device 18 may include an opening or side port 58 through which a therapeutic element 20 may be extended or retracted. For example, the therapeutic element 20 may be a conductive wire (such as Nitinol) or a secondary shaft including one or more electrodes. The therapeutic element 20 may be slidably disposed within the device shaft 36. FIG. 8A shows the treatment device 18 being advanced through the subclavian artery 14 in a collapsed or low-profile configuration, wherein the occlusion element 24 is unexpanded and the therapeutic element 20 is retracted within the device shaft. FIG. 8B shows the treatment device 18 as disposed at a neuromodulation location, wherein the occlusion element 24 is expanded or deployed and the therapeutic element 20 is extended through the side port 58 and in contact with or proximate the wall of the subclavian artery 14. The occlusion element 24 may be distal from the therapeutic element 20, such that neuromodulation occurs upstream of the occlusion element 24. The direction of blood flow within the subclavian artery 14 is depicted with arrow 56. Although not shown, the device 18 shown in FIGS. 8A and 8B may alternatively be positioned within the vertebral artery 16, while still preventing movement of emboli downstream of the occlusion element 24.

Instead of the retractable therapeutic element 20 shown in FIGS. 8A and 8B, the fourth embodiment of a treatment device 18 shown in FIG. 9 includes an expandable therapeutic element 20, such as that shown and described in FIGS. 4A and 4B. Further, although not shown, the device 18 shown in FIGS. 8A and 8B may alternatively be positioned within the vertebral artery 16, while still preventing movement of emboli downstream of the occlusion element 24.

### Selected Examples of Treatment Procedures for Spinal Neuromodulation

Treatment procedures for spinal neuromodulation in accordance with embodiments of the present technology may include applying a treatment modality at one or more treatment locations proximate a structure having a relatively high concentration of nerves. In some embodiments, for example, at least one treatment location may be proximate a portion of a subclavian artery 14, a vertebral artery 16, a junction 26 between a subclavian artery 14 and a vertebral artery 16, a vascular structure proximate one or more dorsal root ganglia of renal nerves, and/or another suitable structure. For example, the stellate ganglia may be targeted if neuromodulation of primarily efferent nerves is desired. Neuromodulation of the stellate ganglia can be expected to have beneficial effect in the treatment of conditions such as Raynaud's phenomenon, hyperhidrosis, and cardiac arrhythmias. Other conditions affected by neuromodulation of the stellate ganglia may include certain chronic pain disorders and hot flashes. Conversely, the dorsal root ganglia of the renal nerves may be targeted if neuromodulation of primarily afferent nerves is desired. Neuromodulation of the dorsal root ganglia of the renal nerves can be expected to have a beneficial effect in the treatment of conditions such as hypertension and high blood pressure.

As described herein, the treatment device 18 may be configured for use with a variety of treatment modalities, such as cryotherapeutic, electrode-based, transducer-based, chemical-based, or another suitable treatment modality. Further, the therapeutic element 20 may be configured to radially expand into a deployed state at the treatment location. Further, the therapeutic element 20 may be configured to create a single lesion or a series of lesions, e.g., overlapping or non-overlapping. In some embodiments, the lesion or pattern of lesions may extend around generally the entire circumference of the vessel, but may still be non-circumferential at longitudinal segments or zones along a lengthwise portion of the vessel. This may facilitate precise and efficient treatment with a lour possibility of vessel stenosis. In other embodiments, the therapeutic element 20 may be configured cause a partially-circumferential lesion, a fully-circumferential lesion at a single longitudinal segment or zone of the vessel, or a helically patterned lesion (for example, a lesion with either discrete lesions in a helical pattern or a continuous helical lesion). During treatment, the therapeutic element 20 may be configured for partial or full occlusion of a vessel. Partial occlusion may be useful, for example, to reduce ischemia, while full occlusion may be useful, for example, to reduce interference (e.g., warming or cooling) caused by blood flow through the treatment location. In some embodiments, the therapeutic element 20 may be configured to cause therapeutically-effective neuromodulation (e.g., using ultrasound energy) without contacting a vessel wall.

A variety of suitable treatment locations are possible in and around the junction 26, the subclavian artery 14, the vertebral artery 16, vascular structures proximate one or more dorsal roots of renal nerves, the stellate ganglia 10, and/or other suitable structures. Furthermore, a treatment procedure may include treatment at any suitable number of treatment locations, e.g., a single treatment location, two treatment locations, or more than two treatment locations. In some embodiments, different treatment locations may correspond to different portions of the junction 26, the subclavian artery 14, the vertebral artery 16, vascular structures proximate one or more dorsal roots of renal nerves, stellate ganglia 10, and/or another suitable structures proximate tissue having relatively high concentrations of sympathetic nerves extending to organs targeted for neuromodulation. The shaft 36 of the device may be steerable (e.g., via one or more pull wires) and may be configured to move the therapeutic element 20 between treatment locations. At each treatment location, the therapeutic element 20 may be activated to cause modulation of nerves proximate the treatment location, as described hereinabove.

The therapeutic element 20 may be positioned at a treatment location within the junction 26, for example, via a catheterization path including a femoral artery, the aorta, and a subclavian artery 14, but other suitable catheterization paths may be used, e.g., a radial or brachial catheterization path. Catheterization may be guided, for example, using imaging, e.g., magnetic resonance, computed tomography, fluoroscopy, ultrasound, intravascular ultrasound, optical coherence tomography, or another suitable imaging modality. The therapeutic element 20 may be configured to accommodate the anatomy of the junction 26, the subclavian artery 14, the vertebral artery 16, a vascular structure proximate dorsal roots of renal nerves, stellate ganglia 10, and/or another suitable structure. For example, the therapeutic element 20 may include a balloon configured to inflate to a size generally corresponding to the internal size of the junction 26, the subclavian artery 14, the vertebral artery 16, a vascular structure proximate one or more dorsal roots of renal nerves, stellate ganglia 10, and/or another suitable structure.

It will be understood that other treatment procedures for spinal neuromodulation in accordance with embodiments of the present technology are also possible.

Methods of neuromodulating, for example, the stellate ganglia or the dorsal root ganglia of the renal nerves in accordance with embodiments of the present technology are expected to improve one or more measurable physiological parameters in patients corresponding to at least one condition associated with sympathetic activity (e.g., overactivity or hyperactivity) in a targeted organ (e.g., the heart or at least one kidney) and/or central sympathetic activity (e.g., overactivity or hyperactivity). With respect to ventricular arrhythmia, modulation of the stellate ganglia in accordance with embodiments of the present technology is expected to reduce the severity and/or frequency of arrhythmia episodes in a patient. With respect to central sympathetic activity (e.g., overactivity or hyperactivity), for example, modulation of one or more dorsal root ganglia of renal nerves in accordance with embodiments of the present technology is expected to reduce muscle sympathetic nerve activity (e.g., at least about 10%) and/or whole body norepinephrine spillover (e.g., at least about 10%) in patients, such as the norepinephrine spillover from sympathetic nerves innervating blood vessels. These and other clinical effects are expected to be detectable immediately after a treatment procedure or after a delay, e.g., of 1, 2, or 3 months. For example, a central sympathetic system activity characteristic of interest may be measured in a patient before the treatment procedure, such as muscle sympathetic nerve activity or whole body norepinephrine spillover. During or after the treatment procedure, the characteristic of interest may again be measured within the patient and compared to the pre-treatment measurement. Then, a value of change in central sympathetic activity with regard to the central sympathetic system activity characteristic of interest may be assessed or quantified based at least in part on the comparison. As a non-limiting example, a predetermined target value of change may be determined, such as reduction of muscle sympathetic nerve activity or whole-body norepinephrine spillover of at least approximately 10% from the pre-treatment measurement. Additionally or alternatively, a threshold value may be defined for one or more non-SNS activity characteristics, such as ECG analysis including QT interval and heart rate, echocardiography analysis including cardiac structure and function, frequency of atrial arrhythmia, frequency of ventricular ectopy, heart rate variability, cardiac norepinephrine spillover, blood pressure, differential effects of left versus right bilateral stellate ganglion neuromodulation, measure of arterial blood flow and/or vascular compliance in the arm, perceived pain, symptom improvements, etc. Specifically, a predetermined target value of change in the treatment of Raynaud's phenomenon may include reduction in the occurrence and/or severity of ulceration (for example, digital ulceration), pain, vasospasm, and vasoconstriction. Also, a predetermined target value of change in the treatment of hyperhidrosis may include reduction in the occurrence and/or severity of excessive sweating.

### EXAMPLES

1. A method for neuromodulation within a patient, the method comprising:
intravascularly positioning a medical device including a therapeutic element in the patient proximate a spinal ganglion; and
activating the therapeutic element to modulate the spinal ganglion.

2. The method of example 1, wherein the therapeutic element is positioned proximate a junction of the subclavian artery and the vertebral artery.

3. The method of example 1 or example 2, further comprising:
determining a first central sympathetic system activity characteristic value within the patient before modulation of the spinal ganglion;
determining a second central sympathetic system activity characteristic value within the patient during or after modulation of the spinal ganglion;
comparing the first value to the second value; and
calculating a value of change in central sympathetic activity with respect to the central sympathetic system activity characteristic based at least in part on the comparison between the first value and the second value.

4. The method of any of examples 1-3, wherein the spinal ganglion is the stellate ganglion.

5. The method of examples 1-4, wherein the method is used to treat patients having been diagnosed with a condition selected from the group consisting of Raynaud's phenomenon, hyperhidrosis, and cardiac arrhythmia.

6. The method of examples 1, 2, 4, and 5, further comprising:
determining a first body system activity characteristic value within the patient before modulation of the spinal ganglion;
determining a second body system activity characteristic value within the patient during or after modulation of the spinal ganglion;
comparing the first value to the second value; and
calculating a value of change in central sympathetic activity with respect to the body system activity characteristic based at least in part on the comparison between the first value and the second value.

7. The method of example 6, wherein the body system activity characteristic is at least one of QT interval, heart rate, cardiac structure, cardiac function, frequency of atrial arrhythmia, frequency of ventricular ectopy, heart rate variability, cardiac norepinephrine spillover, blood pressure, atrial blood flow in the arm, vascular compliance in the arm, perceived pain, occurrence of digital ulceration, severity of digital ulceration, vasospasm, vasoconstriction, occurrence of excess sweating, and severity of excess sweating.

8. The method of any of examples 3-5, wherein the central sympathetic system activity characteristic is at least one of muscle sympathetic nerve activity and whole-body norepinephrine spillover, and a predetermined target value of change in central sympathetic activity is determined, the predetermined target change being a reduction of muscle sympathetic nerve activity or the whole-body norepinephrine spillover of at least approximately 10% in a period of time after modulating the stellate ganglion.

9. The method of any of examples 1-8, wherein modulating the stellate ganglion includes at least one of at least partially disrupting stellate ganglion nerve function and at least partially regulating stellate ganglion nerve function.

10. The method of any of examples 1-9, wherein modulating the stellate ganglion includes thermally modulating the stellate ganglion.

11. The method of example 10, wherein thermally modulating the stellate ganglion includes delivering at least one of radiofrequency energy, optical energy, ultrasound energy, microwave energy, pulsed current energy, direct heat energy, high intensity focused ultrasound energy, or combinations thereof from the therapeutic element to the stellate ganglion, and cryotherapeutically cooling the stellate ganglion with the therapeutic element.

12. The method of example 10 or example 11, wherein thermally modulating the stellate ganglion includes ablating the stellate ganglion.

13. The method of any of examples 1-12, wherein the therapeutic element is selectively adjustable between a delivery configuration and a deployed configuration, and modulating the stellate ganglia includes adjusting the therapeutic element from the delivery configuration to the deployed configuration at a treatment location proximate the junction of the subclavian artery and vertebral artery.

14. The method of any of examples 1-13, wherein the therapeutic element includes an elongate member that is at least partially helical in the deployed configuration, the elongate member having one or more electrodes configured to deliver radiofrequency energy.

15. The method of any of examples 1-14, wherein the therapeutic element includes a balloon that is at least partially inflated in the deployed configuration.

16. The method of any of examples 1-15, wherein the therapeutic element is configured for at least one of the application of radiofrequency energy to the stellate ganglion, the application of a therapeutic compound to the stellate ganglion, and cryotherapeutic cooling of the stellate ganglion.

17. The method of any of examples 1-16, wherein the medical device further includes one of an occlusion element located distal from the therapeutic element and an occlusion element located proximal from the therapeutic element.

18. A method of treating a human patient diagnosed with cardiac arrhythmia, the method comprising:
positioning a medical device including a therapeutic element proximate a junction between a subclavian artery and a vertebral artery in a patient; and
at least partially inhibiting neural activity in nerves proximate the junction with the therapeutic element.

19. The method of example 18, wherein at least partially inhibiting neural activity in nerves proximate the junction includes at least partially inhibiting neural activity in a stellate ganglion.

20. The method of example 18 or example 19, further comprising:
determining a first cardiac activity characteristic value within the patient before the at least partial inhibition of the neural activity;
determining a second cardiac activity characteristic value within the patient during or after the at least partial inhibition of the neural activity;
comparing the first value to the second value; and
calculating a value of change in central sympathetic system activity with respect to the cardiac activity characteristic based at least in part on the comparison between the first value and the second value,
the cardiac activity characteristic being at least one of severity of cardiac arrhythmia episodes within the patient and frequency of cardiac arrhythmia episodes in the patient.

21. The method of example 20, wherein the value of change in central sympathetic system activity with respect to the cardiac system activity characteristic is satisfactory when the second value is less than the first value.

22. The method of any of examples 18-21, wherein the therapeutic element is selectively transitionable between a delivery configuration and a deployed configuration, and at least partially inhibiting neural activity includes transitioning the therapeutic element from the delivery state to the deployed state at a treatment location proximate the junction.

23. The method of any of examples 18-22, wherein the therapeutic element en includes one of an elongate member that is at least partially helical in the deployed state and a balloon that is at least partially inflated in the deployed state.

24. The method of any of examples 18-23, wherein at least partially inhibiting neural activity includes thermally modulating the nerves.

25. The method of example 24, wherein thermally modulating the nerves includes delivering radiofrequency energy to the nerves.

26. The method of example 24 or example 25, wherein thermally modulating the nerves includes ablating the nerves.

27. The method of any of examples 18-26, wherein the medical device further includes an occlusion element located distal from the therapeutic element.

28. A method for treating a human patient diagnosed with Raynaud's phenomenon or hyperhidrosis, the method comprising:
positioning a medical device including a therapeutic element proximate a junction between a subclavian artery and a vertebral artery in a patient displaying one or more symptom characteristics;
at least partially inhibiting neural activity in a stellate ganglion proximate the junction with the therapeutic element;
determining a first symptom characteristic value within the patient before the at least partial inhibition of neural activity in the stellate ganglion;
determining a second symptom characteristic value within the patient during or after the at least partial inhibition of neural activity in the stellate ganglion;
comparing the first value to the second value; and
calculating a value of change in central sympathetic system activity based at least in part on the comparison between the first value and the second value.

29. The method of example 28, wherein the one or more symptom characteristics include digital ulceration, vasoconstriction, vasospasm, pain, and excessive sweating, and the value of change in central sympathetic system activity with respect to the symptom characteristic value is satisfactory when the second value is less than the first value.

### Conclusion

The above detailed descriptions of embodiments of the present technology are for purposes of illustration only and are not intended to be exhaustive or to limit the present technology to the precise form(s) disclosed above. Various equivalent modifications are possible within the scope of the present technology, as those skilled in the relevant art will recognize. For example, while steps may be presented in a given order, alternative embodiments may perform steps in a different order. The various embodiments described herein and elements thereof may also be combined to provide further embodiments. In some cases, well-known structures and functions have not been shown or described in detail to avoid unnecessarily obscuring the description of embodiments of the present technology.

Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout the disclosure to mean including at least the recited feature(s) such that any greater number of the same feature(s) and/or additional types of other features are not precluded. It will also be appreciated that various modifications may be made to the described embodiments without deviating from the present technology. Further, while advantages associated with certain embodiments of the present technology have been described in the context of those embodiments, other embodiments may also exhibit such advantages, and not all embodiments need necessarily exhibit such advantages to fall within the scope of the present technology. Accordingly, the disclosure and associated technology may encompass other embodiments not expressly shown or described herein.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention.

What is claimed is:

1. A method for neuromodulation within a patient, the method comprising:
intravascularly positioning a medical device including a therapeutic element in the patient proximate a spinal ganglion; and
activating the therapeutic element to modulate the spinal ganglion.

2. The method of claim 1, wherein the therapeutic element is positioned proximate a junction of the subclavian artery and the vertebral artery.

3. The method of claim 2, further comprising:
determining a first central sympathetic system activity characteristic value within the patient before modulation of the spinal ganglion;
determining a second central sympathetic system activity characteristic value within the patient during or after modulation of the spinal ganglion;
comparing the first value to the second value; and
calculating a value of change in central sympathetic activity with respect to the central sympathetic system activity characteristic based at least in part on the comparison between the first value and the second value.

4. The method of claim 3, wherein the spinal ganglion is the stellate ganglion.

5. The method of claim 4, wherein the central sympathetic system activity characteristic is at least one of muscle sympathetic nerve activity and whole-body norepinephrine spillover, and a predetermined target value of change in central sympathetic activity is determined, the predetermined target change being a reduction of muscle sympathetic nerve activity or the whole-body norepinephrine spillover of at least approximately 10% in a period of time after modulating the stellate ganglion.

6. The method of claim 4, wherein modulating the stellate ganglion includes at least one of at least partially disrupting stellate ganglion nerve function and at least partially regulating stellate ganglion nerve function.

7. The method of claim 6, wherein modulating the stellate ganglion includes chemically modulating the stellate ganglion.

8. The method of claim 7 wherein modulating the stellate ganglion includes thermally modulating the stellate ganglion.

9. The method of claim 8, wherein thermally modulating the stellate ganglion includes delivering at least one of radiofrequency energy, optical energy, ultrasound energy, microwave energy, pulsed current energy, direct heat energy, high intensity focused ultrasound energy, or combinations thereof from the therapeutic element to the stellate ganglion, and cryotherapeutically cooling the stellate ganglion with the therapeutic element.

10. The method of claim 9, wherein thermally modulating the stellate ganglion includes ablating the stellate ganglion.

11. The method of claim 9, wherein the therapeutic element is selectively adjustable between a delivery configuration and a deployed configuration, and modulating the stellate ganglia includes adjusting the therapeutic element from the delivery configuration to the deployed configuration at a treatment location proximate the junction of the subclavian artery and vertebral artery.

12. The method of claim 11, wherein the therapeutic element includes an elongate member that is at least partially helical in the deployed configuration, the elongate member having one or more electrodes configured to deliver radiofrequency energy.

13. The method of claim 11, wherein the therapeutic element includes a balloon that is at least partially inflated in the deployed configuration.

14. The method of claim 13, wherein the therapeutic element is configured for at least one of the application of radiofrequency energy to the stellate ganglion, the application of a therapeutic compound to the stellate ganglion, and cryotherapeutic cooling of the stellate ganglion.

15. The method of claim 5, wherein the medical device further includes one of an occlusion element located distal from the therapeutic element and an occlusion element located proximal from the therapeutic element.

16. The method of claim 2, wherein the spinal ganglion is the stellate ganglion, the method further comprising:
determining a first body system activity characteristic value within the patient before modulation of the spinal ganglion;
determining a second body system activity characteristic value within the patient during or after modulation of the spinal ganglion;
comparing the first value to the second value; and
calculating a value of change in central sympathetic activity with respect to the body system activity characteristic based at least in part on the comparison between the first value and the second value.

17. The method of claim 16, wherein the body system activity characteristic is at least one of QT interval, heart rate, cardiac structure, cardiac function, frequency of atrial arrhythmia, frequency of ventricular ectopy, heart rate variability, cardiac norepinephrine spillover, blood pressure, atrial blood flow in the arm, vascular compliance in the arm, perceived pain, occurrence of digital ulceration, severity of digital ulceration, vasospasm, vasoconstriction, occurrence of excess sweating, and severity of excess sweating.

18. A method of treating a human patient diagnosed with cardiac arrhythmia, the method comprising:
positioning a medical device including a therapeutic element proximate a junction between a subclavian artery and a vertebral artery in a patient; and
at least partially inhibiting neural activity in nerves proximate the junction with the therapeutic element.

19. The method of claim 18, wherein at least partially inhibiting neural activity in nerves proximate the junction includes at least partially inhibiting neural activity in a stellate ganglion.

20. The method of claim 19, further comprising:
determining a first cardiac activity characteristic value within the patient before the at least partial inhibition of the neural activity;
determining a second cardiac activity characteristic value within the patient during or after the at least partial inhibition of the neural activity;
comparing the first value to the second value; and
calculating a value of change in central sympathetic system activity with respect to the cardiac activity characteristic based at least in part on the comparison between the first value and the second value,
the cardiac activity characteristic being at least one of severity of cardiac arrhythmia episodes within the patient and frequency of cardiac arrhythmia episodes in the patient.

21. The method of claim 20, wherein the value of change central sympathetic system activity with respect to the cardiac system activity characteristic is satisfactory when the second value is less than the first value.

22. The method of claim 20, wherein the therapeutic element is selectively transitionable between a delivery configuration and a deployed configuration, and at least partially inhibiting neural activity includes transitioning the therapeutic element from the delivery state to the deployed state at a treatment location proximate the junction.

23. The method of claim 21, wherein the therapeutic element includes one of an elongate member that is at least partially helical in the deployed state and a balloon that is at least partially inflated in the deployed state.

24. The method of claim 22, wherein at least partially inhibiting neural activity includes thermally modulating the nerves.

25. The method of claim 24, wherein thermally modulating the nerves includes delivering radiofrequency energy to the nerves.

26. The method of claim 25, wherein thermally modulating the nerves includes ablating the nerves.

27. The method of claim 8, wherein the medical device further includes an occlusion element located distal from the therapeutic element.

28. A method for treating a human patient diagnosed with Raynaud's phenomenon or hyperhidrosis, the method comprising:
positioning a medical device including a therapeutic element proximate a junction between a subclavian artery and a vertebral artery in a patient displaying one or more symptom characteristics;
at least partially inhibiting neural activity in a stellate ganglion proximate the junction with the therapeutic element;
determining a first symptom characteristic value within the patient before the at least partial inhibition of neural activity in the stellate ganglion;
determining a second symptom characteristic value within the patient during or after the at least partial inhibition of neural activity in the stellate ganglion;
comparing the first value to the second value; and
calculating a value of change in central sympathetic system activity based at least in part on the comparison between the first value and the second value.

29. The method of claim 28, wherein the one or more symptom characteristics include digital ulceration, vasoconstriction, vasospasm, pain, and excessive sweating, and the value of change in central sympathetic system activity with respect to the symptom characteristic value is satisfactory when the second value is less than the first value.
